(19) United States
(12) Patent Application Publication
(10) Pub. No.: US 2003/0150475 A1
(43) Pub. Date: Aug. 14, 2003

(54) Method and apparatus for sanitizing reusable articles

(76) Inventors: Lorne Abrams, Windermere, FL (US); Jay Palmer, Orlando, FL (US); Letitia Strowbridge, Buford, GA (US); Keith B. Nowack, Acworth, GA (US); Jorge L. Rodriguez, Winter Garden, FL (US)

Correspondence Address: Mark D. Passler, Akerman, Senterfitt & Eidson, P.A., P.O. Box 3188, West Palm Beach, FL, 33402-3188, US

(21) Appl. No.: 10/074,111

(22) Filed: Feb. 11, 2002

(51) Int. Cl. B08B 7/00

(52) U.S. Cl. 134/1; 134/18

(57) ABSTRACT

An apparatus for sanitizing articles includes a conveyor for conveying articles through the apparatus, and an irradiation chamber surrounding the conveyor. A plurality of UV lamps is arranged in the irradiation chamber to irradiate with UV light articles conveyed through the irradiation chamber on the conveyor. The intensity and dose of UV light may be varied to achieve a desired level of sanitization. A method of sanitizing articles for re-use includes checking articles for cleanliness, and washing only articles which are discovered to be below a predetermined standard of cleanliness. Articles which are discovered to be above a predetermined standard of cleanliness are irradiated with UV light in order to sanitize such articles without washing. Articles which are below the predetermine standard of cleanliness may be washed in a traditional wash system prior to being irradiated with UV light.

### CROSS-REFERENCE TO RELATED APPLICATIONS

Not applicable.

### STATEMENT REGARDING FEDERALLY-SPONSORED RESEARCH

Not applicable.

### BACKGROUND OF THE INVENTION

1. Field of the Invention

The present invention relates generally to sanitizing methods and systems and more particularly methods and systems for sanitizing articles such as containers.

2. Background of the Invention

Containers, including returnable plastic containers, also known as RPCs, are a growing market in the food supply chain. RPCs are used primarily by the produce industry for transport of various foodstuffs from the grower/manufacturer through distribution channels to the retailer. An RPC is a rigid collapsible plastic crate with inwardly or outwardly folding walls that are hinged to a base by a reversing hinge pin. The sides interlock at the corners to form a crate for the storage and transportation of produce. The side walls and base are specifically designed for product ventilation; thus they contain an intricate web of cross column and lateral support members to enable stacking. RPCs are available in many different sizes and are used in a wide range of applications, including food contact and non-food contact applications.

After use, RPCs are returned from the user to a processing center for cleansing and refurbishment if necessary. The method of cleansing most currently used is hot water washing, typically using a chlorine or an ammonia chloride water based sanitizing agent or detergent dissolved into the wash or rinse water. The RPCs are loaded onto a conveyor and conveyed through a wash tunnel where they are sprayed and washed with hot water and a chemical emulsion mixture, typically at an elevated temperature, to aid in the removal of soil and residual food. The hot water and detergent wash removes the soil and food particles, and the chemical mixture includes a caustic solution which kills bacteria colonies on the container. This process is designed to achieve the 5 log reduction in bacterial concentration required to meet FDA requirements for a sanitized RPC. It is difficult to consistently meet the FDA 5 log requirement using the wet wash method because the temperature fluctuates between thermostatically controlled upper and lower limits and the amount of chemical applied can vary due to diluted dosing rates and variable water flows. As a result, the reliability of the process is dependent upon the accuracy of the process control capability of the wash system and the chemical titration method used by the wash line operator. An alternative method of washing involves the submersion of the RPCs in a water and detergent bath, which is subject to the same chemical conditions described above. In both methods, the RPCs must then be subject to a drying cycle, which can last up to seven days, and can involve heat, forced air, high velocity compressed air or water evaporation. The washing and drying of RPC crates is very costly, and the necessary washing and drying equipment is expensive and labor intensive to maintain. RPCs that are dispatched wet or damp to users possess a greater opportunity for contamination of a previously sanitized container, for example, due to the growth of mold and mildew. It is clear that the wet ‘sanitizing’ process provides increased possibilities for the harboring and reproduction of dangerous microorganisms either through inadequate wash methodologies, chemical concentrations that are too dilute or improper and insufficient drying of RPC units.

A study of the returning pool of RPCs has indicated that on average between 34% and 50% of the used RPCs contain contaminants such as soil and/or food residue that would require washing. The remaining population of used containers does not contain objectionable amounts of soils and residue so as to require washing, but it is desirable to sanitize such containers before reuse. It is also desirable to provide a method of sanitizing articles that reduces the amount of washing and drying that is necessary prior to reuse in order to reduce the amount of water and detergents consumed and to eliminate the added complexities which the wet washing process contributes towards the requirement for a 5 log reduction in bacteria for the sanitization of RPC containers.

It is desirable to sanitize many other types of articles, not just containers such as RPCs. There are also many industries where sanitization of articles is desirable, including health care, laboratories and other scientific establishments, manufacturing (such as the manufacture of pharmaceuticals, semiconductor chips, and medical devices, among others). Suitable articles for sanitization include transportation containers, packaging materials, tools, parts of manufacturing machinery, clothing, footwear, protective gear, and many ancillary devices. Such articles may not require a 5 log reduction in bacteria, or may require a greater degree of sanitization, depending on their intended uses.

U.S. Pat. No. 5,809,739 to Eno is directed to a filling machine having a system to aid in sterilizing surfaces of single-use cartons, such as the cartons used to supply milk and fruit juices to end consumers. The packaging machine of this patent includes a sterilization station where the cartons to be sanitized are subject to a hydrogen peroxide spray followed by UV radiation by a light assembly, which is used to sterilize both the interior and exterior of the carton prior to filling with product. The irradiation chamber includes reflecting surfaces to achieve sterilization by reflecting the UV radiation to all surfaces of the carton.

U.S. Pat. No. 5,744,904 to Castberg et al. is directed to a sterilization method in which items or materials can be sterilized with an UV laser, an infrared device (not limited to a laser) and/or a hydrogen peroxide solution. The UV laser can be used alone or in combination with the infrared device and/or the hydrogen peroxide solution. The sterilization method is described as being for paperboard packaging material that may be coated with a thin polymer layer. Such thermoplastic surfaces are stated to be particularly sensitive to heat, and care must be taken not to melt the thermoplastic with the laser.

U.S. Pat. No. 5,326,542 to Sizer et al., U.S. Pat. No. 6,066,081 to Bachner and U.S. Pat. No. 6,094,887 to Swank et al. also relate to UV sterilization of food cartons prior to their filling with liquids.

The known UV sterilization devices all seek to sterilize single-use cartons, which are typically formed of a thin paperboard material. The paperboard may be coated with an aluminum foil and/or a thin polymer layer. There has been no known attempt at sanitization of reusable articles such as plastic containers, which are thicker and tend to have more complicated shapes than cartons. Further, the known prior art teaches that UV light on its own is not capable of adequately sterilizing articles.

### SUMMARY OF THE INVENTION

One aspect of the present invention relates to an apparatus for sanitizing articles. The apparatus includes a conveyor for conveying articles through the apparatus, and an irradiation chamber surrounding the conveyor. A plurality of UV lamps is arranged in the irradiation chamber to irradiate with UV light articles conveyed through the irradiation chamber on the conveyor.

The conveyor may continuously convey articles through the irradiation chamber. In one arrangement, the UV lamps may be arranged above and below the conveyor. Alternatively, the lamps may be arranged on opposing sides of a hanger style conveyor. The conveyor may have apertures to allow UV light to irradiate the bottom of articles placed thereon. The conveyor may be a chain conveyor, and may include a plurality of chain conveyors, each having chains located at different widths across the conveyor effectively allowing for the transfer of the RPC container from one chain position to an alternate chain position. Each of the chain conveyors may have two or more chains. In an alternate arrangement, the conveyor may be an overhead conveyor and may include a plurality of hanging clips to clasp the RPC as it is transferred through the system.

The plurality of UV lamps may be arranged in an ordered array. The array of UV lamps may include lamps of different lengths. The UV lamps may be elongated and define an axis, and at least one of the UV lamps may be arranged with its axis perpendicular to the direction of the conveyor. At least one of the UV lamps may be arranged with its axis at an angle to the direction of the conveyor.

In one arrangement, at least one of the UV lamps preferably includes a parabolic reflector. The reflector may be water-cooled, or may be cooled by a chiller, an air conditioning unit, a high velocity air flow or by any other suitable means. The UV lamps may be medium pressure mercury vapor arc lamps.

Another aspect of the invention relates to a method of sanitizing articles for re-use. The method includes checking articles for cleanliness, and washing only articles which are discovered to be below a predetermined standard of cleanliness. The washing process may involve a chemical application. Articles which are discovered to be above a predetermined standard of cleanliness are irradiated with UV light in order to sanitize such articles without washing.

The washed articles may be irradiated with UV light after the washing step is completed. The irradiation may take places in a sanitization chamber while the articles are continuously moved through the chamber by a conveyor. The speed of the conveyor may be variable (and hence the exposure time of the article in the chamber), and/or the intensity of the UV light may be variable, in order to vary the total UV dose given to the articles. The UV dose given to articles may be measured and/or monitored and confirm by periodically passing a UV radiometer through the chamber.

Notably, an advantage of the sanitization method and apparatus according to the invention are the complete or near elimination of the use of large amounts of water and/or chemicals to clean the articles and a reduction in the amount of time required to dry such articles.

### BRIEF DESCRIPTION OF THE DRAWINGS

There are shown in the drawings embodiments which are presently preferred, it being understood, however, that the invention is not limited to the precise arrangements and instrumentality shown, wherein:

FIG. 1 is a perspective view of a sanitizing system according to the invention;

FIG. 2 is a perspective view of a sanitizing system according to the invention in an open position;

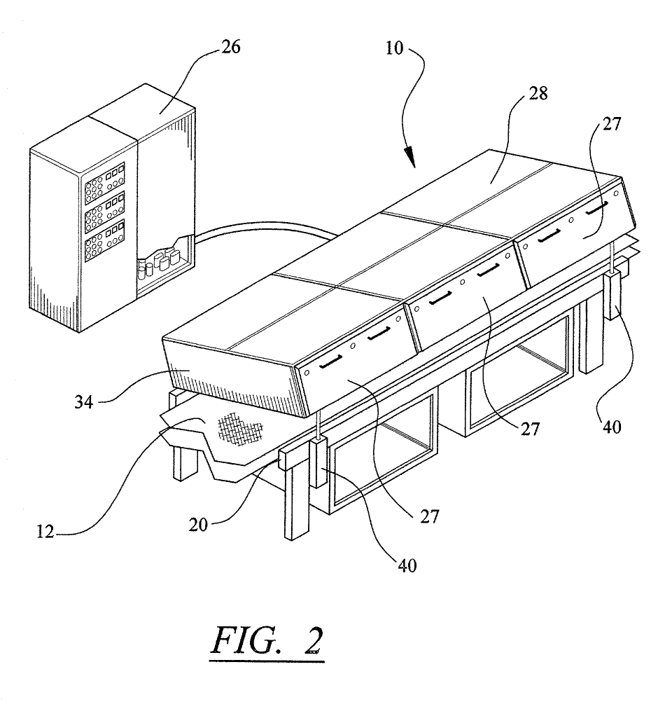

FIG. 3 is a front, part cross-sectional view of a sanitizing system according to the invention showing interior features in dashed lines;

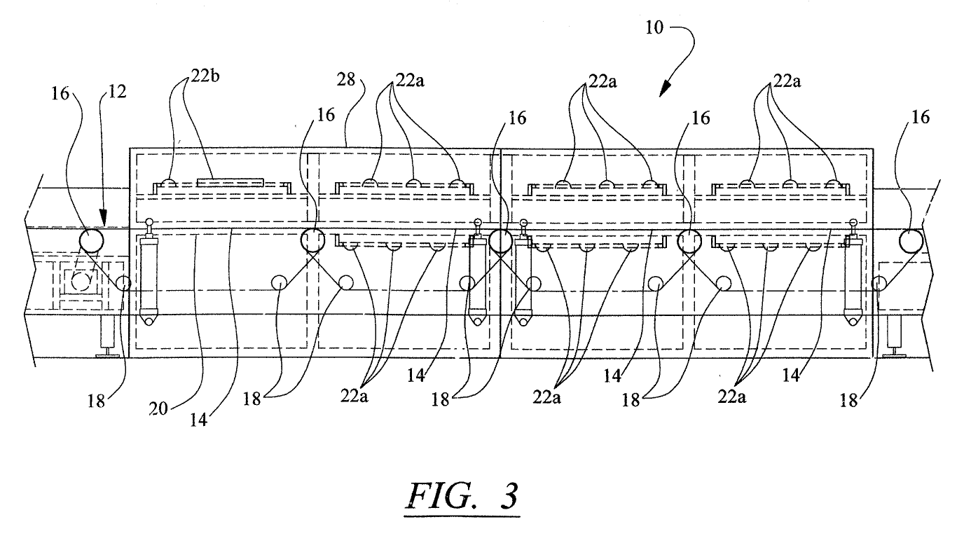

FIG. 4 is a top, part cross-sectional view of a sanitizing system according to the invention showing interior features in dashed lines;

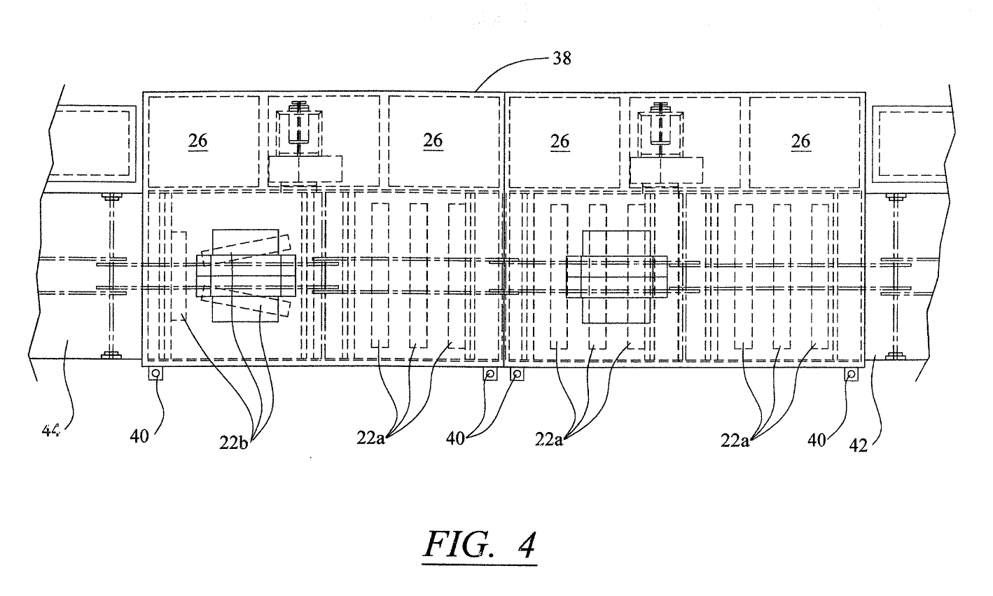

FIG. 5 is an end view of a sanitizing system according to the invention in an open position showing interior features in dashed lines;

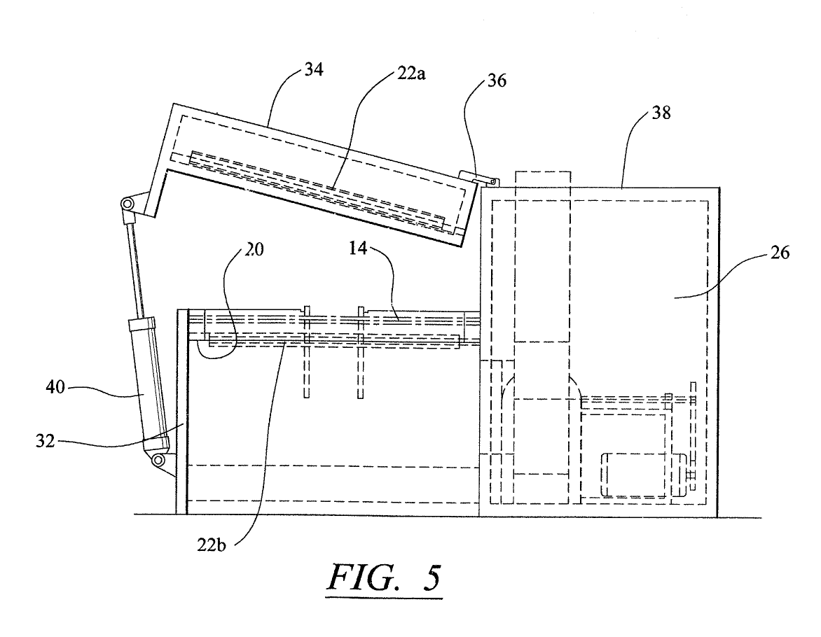

FIG. 6 is a perspective view of an article which can be sanitized according to the invention;

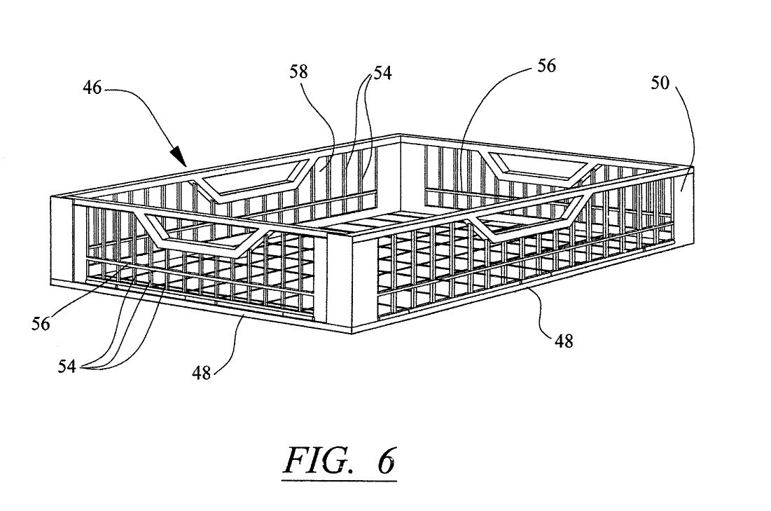

FIG. 7 is a view of a UV lamp which can be used to sanitize articles according to the invention; and

FIG. 8 is a flowchart illustrating a method according to the invention.

### DETAILED DESCRIPTION OF EMBODIMENTS OF THE INVENTION

The drawings and the following description relate to one embodiment of a sanitizing system 10 suitable for sanitizing articles including containers such as Returnable Plastic Containers (RPCs). It will be appreciated that the methods and systems are suitable for any article, and that references to reusable articles, containers and RPCs should be treated as exemplary only. In particular, containers and packaging materials such as crates, pallets, boxes, trays, bowls, tubs, mail bins and other containers not limited to the food industry are also suitable for sanitization with the method and system of the invention. Other articles such as produce harvesting equipment, food processing equipment, food preparation equipment, food serving dishes, plates, flatware, and stemware, portable preparation surfaces such as chopping boards and pastry boards are also suitable for sanitization with the method and system of the invention.

The illustrated sanitizing system 10 according to the invention includes a conveying system 12 that can be formed of a plurality of interconnected chain conveyors 14, powered by drive shafts 16 and having rollers 18. The chain conveyors 14 may be formed of any suitable material, such as plastic or metal such as stainless steel, plated steel, aluminum and the like. A belt or mesh conveyor system can also be utilized. Alternatively, an overhead hanger style conveyor system may be used. The sanitizing system 10 can also include at least one UV lamp for irradiating articles on the conveyor. The chain conveyors 14 can include chains located at differing widths across the conveying system 12 so that an article placed on the conveyor does not always rest on the same points, thus avoiding shadowed areas of the article that cannot be penetrated by UV light. In the illustrated embodiment, four chain conveyors are used, each formed of two chains. It will be appreciated however that any suitable form and number of conveyors may be employed in the invention. The conveying system 12 may be located in a shallow stainless steel trough 20, which aids in placement and control of articles placed on the conveyor.

Two adjacent chain conveyors 14 can be tied together through common drive shafts 16 and can be driven by a motor (not shown). A suitable motor for certain arrangements is a single 3HP gear motor. The gear motor can be equipped with a variable frequency drive (VFD) or a variable speed drive (VSD). This enables the speed of the conveyor chain to be manipulated to allow for adjustments in exposure time. As an alternative, a belt conveyor system could be utilized which will allow the article to be turned over by 180 degrees and conveyed through additional UV lamps (in the up-side-down position or otherwise re-oriented, repositioned, or partially opened or collapsed) to assure 100% UV light exposure to all internal and external faces of the article. As another alternative, an overhead conveyor system could be utilized which allows an article to be hung on a clasp fixture and transferred through arrays of UV lamps positioned on opposing sides of the conveyor. Further, automatic UV sensing equipment can be linked to the VFD or VSD via a programmable logic controller thus controlling speed to maintain a predetermined dose of UV energy.

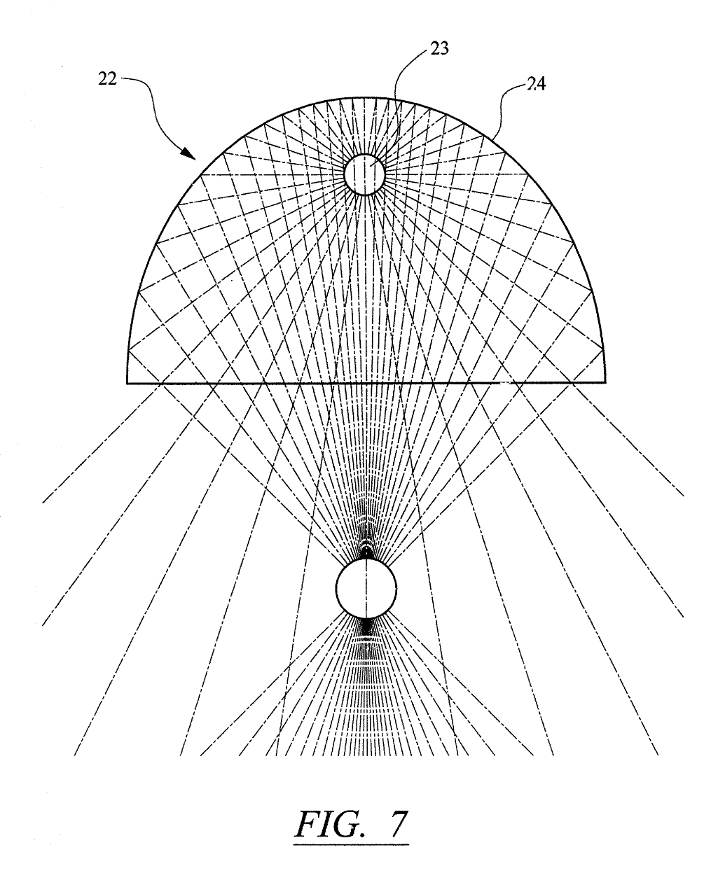

A UV lamp 22 suitable for use in the invention can have a medium pressure mercury vapor source 23, and can emit UV Light in the 250-260 nanometer (nm) range. Alternatively, low pressure or high pressure mercury vapor lamps may be used, and higher or lower wavelengths may also be used. The use of UV light for irradiating and destroying microorganisms is well known. Cells of organisms absorb UV light in the 250-260 nm wavelength range, particularly at around 254 nm. This absorption forms a chemical bond between adjacent thymine nucleotide bases, which distorts the DNA strands and hinders replication of the organism. Other wavelength ranges may be suitable for certain applications. The quantity of cells destroyed depends upon exposure time and dose measured in mW/cm² or mJ/cm². A UV lamp 22 suitable for use in the invention can have a parabolic reflector 24. The parabolic reflector 24 reflects the UV emissions from the lamp 22 at varying angles, as can be seen from FIG. 7, as opposed to a focused light source obtained with a laser or elliptical reflector. However, in certain arrangements a focused light source may be applicable. Other UV lamp sources and reflector shapes may be used. Alternatively, diffusers may be placed in front of the light source. The reflectors 24 may be water cooled, or may be cooled by a chiller, air conditioning unit, a high velocity air flow, or any other suitable means. In one arrangement, heat removed from the lamps by the cooling process may be recovered and applied to the cleaning or preparation process, or may be redirected to traditional washing and drying processes. In certain arrangements, cooling of the reflectors may not be necessary.

The UV lamp 22 can be powered by any suitable power source. In one embodiment, a dual range ballast 26 is employed, which is capable of supplying power to the UV lamp 22 equivalent to 200 watts per inch (wpi) and 300 wpi. This allows for suitable and variable UV dosage, depending on the desired output of the lamps. Alternatively, lamps may be powered by an adjustable (rheostat-type) ballast allowing infinite adjustment of power between 0 watts and the lamp maximum. It will be appreciated that any suitable power source may be used, to generate any suitable intensity of UV light.

The UV lamps 22 can be arranged in an ordered array around the 12. The exemplary arrangement illustrated in the drawings uses 21 elongated UV lamps 22, comprising 18 lamps 22a having a length of 48″, and 3 lamps 22b having a length of 24″. Other sizes, quantities and arrangements of lamps can be employed, depending on the articles to be sanitized, the degree of sanitization required, and many other factors. Each lamp 22 can be positioned to provide the maximum exposure of UV light to all surfaces of an article passing on the conveyor 12. Nine of the 48″ lamps 22a can be located above the conveyor in banks of three arranged perpendicularly to the direction of the conveyor to reflect UV light down onto the conveyor, and nine of the 48″ lamps 22a can be located in banks of three underneath the conveyor 12 to reflect UV light upwardly onto the conveyor 12. The upper and lower banks of three UV lamps 22 may alternate, and can be positioned so that light from one bank overlaps light from the next bank. All three 24″ lamps 22b can be located above the conveyor 12 at an exit end thereof and can be angled to ensure penetration of UV energy into hard to reach areas of an article, such as the hinges, hinge pins and corners of an RPC. It will be appreciated that any suitable shape, number and location of lamps may be used. In one arrangement, the lights and components that disseminate the UV light can be strategically positioned or shaped so as to increase the exposure of those areas of the articles that are generally the most heavily contaminated. Further, reflective surfaces may be situated in certain areas of the apparatus to modify the focal pattern of the ultraviolet waves to ensure that crucial areas of the articles are reached. The lamps may have protective glass barriers at their fronts to protect them from contact with dirt, dust and other foreign matter. The UV lamps may be contained in drawer-style assemblies 27 that can be opened for servicing, cleaning and bulb replacement.

The conveyor 12 and UV lamps 22 can be enclosed by a casing 28 to prevent escape of UV light, forming a sanitization chamber 30. Casing 28 can comprise a lower unit 32 and an upper unit 34. The upper unit 34 can have a hinge 36 along its top, connecting it to chambers 38 for the ballasts 26. The upper unit may be opened by a plurality of pneumatic or hydraulic pistons 40 in order to allow access to the sanitization chamber 30 for maintenance. To the sides of casing 28, the conveyor 12 is joined to an in-feed conveyor 42 and an out-feed conveyor 44 (shown only partially in the drawings).

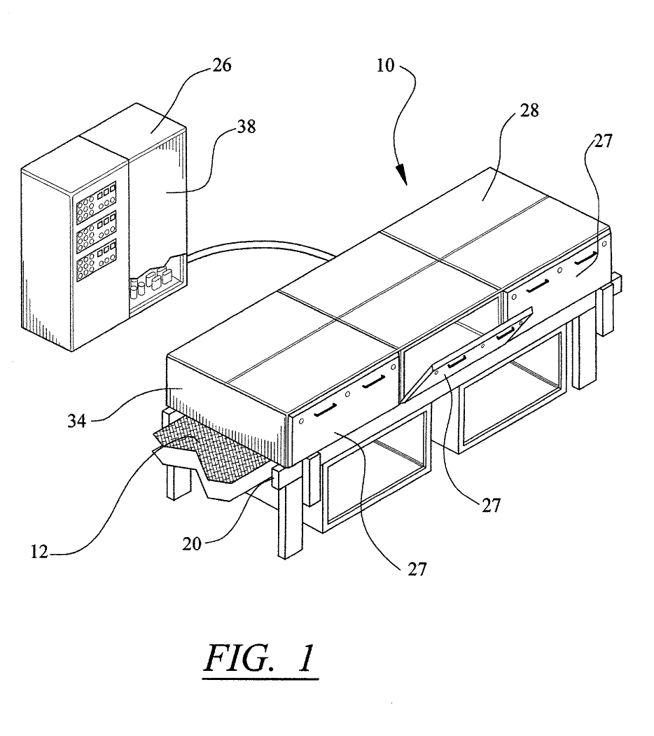

As shown in FIG. 1, an RPC 46, which may be sanitized using the method and system of the invention, is a collapsible plastic crate having a base 48 and walls 50. The walls 50 are hinged to the base 48 through a reversing hinge pin 52. The sides can interlock at the corners to form an upright crate. The side walls 50 and base 48 are specifically designed for product ventilation; thus they contain an intricate web of cross column and lateral support members 54 and 56 to give strength and enable stacking. Ventilation gaps 58 are formed between the support members 54 and 56. RPCs are available in many different sizes, and shapes, and it will be appreciated that RPCs are referred to herein for the purposes of illustration only. The invention is not limited to the sanitization of RPCs or other containers, and it will be appreciated that references herein to RPCs are exemplary only.

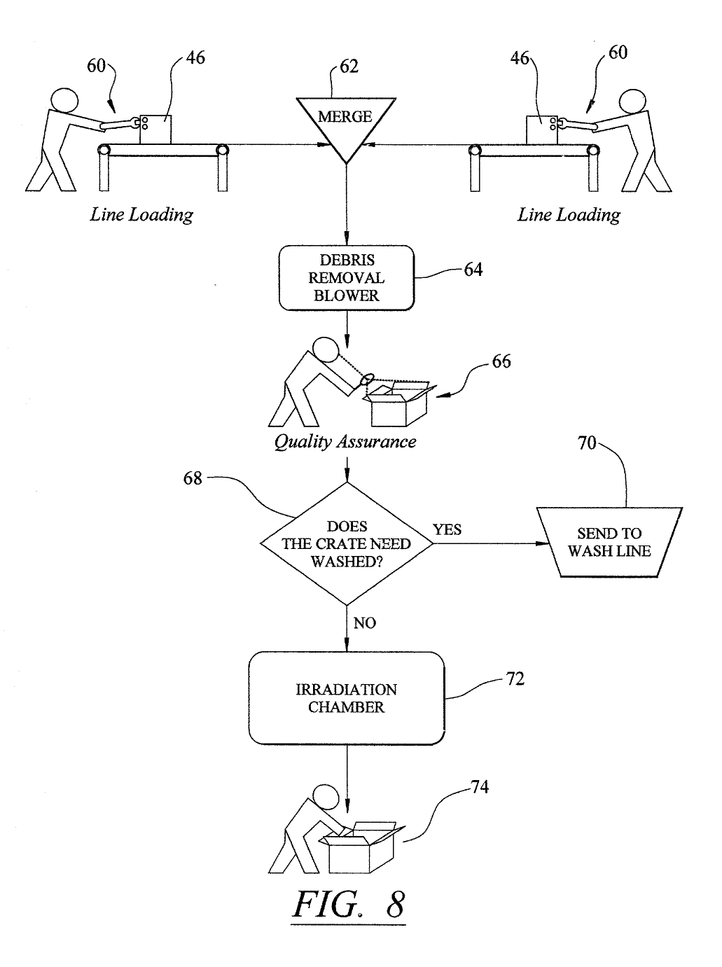

Referring to FIG. 8, in an example sanitizing method according to the invention, after use of RPCs 46 for transporting food products and the like, the RPCs 46 can be returned to a processing center for cleansing and refurbishment if necessary. The processing center can include multiple receiving stations 60, which may be for different sized RPCs, for example. The RPCs 46 can be flattened or opened out at the receiving stations 60, or may be left in an assembled configuration. Other processing may take place at the receiving stations 60, for example, inspection for damage or wear to the RPCs. The RPCs 46 can from different receiving stations 60 can be merged together (62) and can be conveyed through a debris removal blower 64 for initial removal of debris, and can then be conveyed through a quality control station 66 where they are inspected for soil and food residues. If found to be contaminated and in need of washing (68) they can be removed from the conveyor line and sent to a traditional wash system 70. After the contaminated RPCs have been washed, they may be returned to the conveyor for UV sanitizing, or may be omitted from the UV sanitizing process. If found to be free of contaminants, the RPCs 46 can be placed on the in-feed conveyor 42 and conveyed into the sanitization chamber 30 of the present invention for sanitizing 72.

The RPCs 46 can be loaded onto the in-feed conveyor 42 in an open position (sides opened out) or in an assembled position. The in-feed conveyor 42 passes each RPC 46 to the conveyor 12 which runs within a shallow stainless steel trough 20. The trough 20 aids in efficient RPC placement and alignment. As the stainless steel chain conveyors 14 comprise chains having a varying distance between them, shadowing of the bottom of the RPC 46 is eliminated as it passes through the sanitization chamber 30 from one chain conveyor 14 to the next chain conveyor 14. UHMW chain guides may be used to reduce noise and improve conveyor efficiency. It will be appreciated that the chain guides may be of any suitable design or material. Varying the contact points on the bottom of the RPC 46 ensures that the entire bottom of the RPC can be directly exposed to the UV light emitted from lamps 22.

The parabolic reflector 24 of lamps 22 causes reflection of the light in varying directions and enables the UV light to penetrate into the intricate web of cross column and lateral support members 52 and 54 of the ventilated walls 50 and base 48 of an RPC. It also enables UV light to penetrate the hinges and pins 52 that hold the walls 50 to the base 48. This significantly reduces shadowing caused by the sidewall design and leads to a more efficient and effective use of the UV energy, thus ensuring the microorganisms in these areas are destroyed as well as microorganisms present on flat surfaces.

The variable frequency drive (VFD) of chain conveyors 14 enables the speed of the conveyor 12 to be manipulated to allow for adjustments in exposure time. The VFD can give the system a wide range of control parameters to ensure maximum UV dosage and microorganism destruction while not compromising productivity. Further, automatic UV sensing equipment can be linked to the VFD via a programmable logic controller (PLC) thus controlling speed to maintain a predetermined dose of UV energy. The UV sensing equipment may include a UV calibrated radiometer that can be periodically placed on the conveyor 12 and passed through the sanitization apparatus 10 to measure the amount of UV energy incident on the surface of the radiometer (and hence on the surface of the RPCs that are passed through the apparatus). Alternatively, the conveyor can be provided with a UV radiometer to continuously measure the UV dose in the apparatus.

Once the RPC 46 has passed through the sanitization chamber 30, and has undergone the sanitization process 72, it passes to the out-feed conveyor 44 and is classified as sanitized. The RPC can be manually removed (or automatically removed through the use of pick and place style robots or other automated means) from the conveyor 12, segregated by size (through manual or automated methods [using RPC barcodes and/or side wall variances) and prepared for shipment back to users at a shipment station 74. The RPC can be used and sanitized may times during its life.

Sanitized is usually defined by the destruction of 99.999% of all living microorganisms, which is also referred to as a 5-log reduction in bacteria. This may be validated during quality control testing using salmonella or like microorganisms. During testing RPCs are inoculated with the organisms. Viable cells are verified by plate count. The sample may be processed through the sanitization chamber 30 and within 1 minute the inoculated area can be swabbed with a moistened cotton swab and placed in a neutralizing buffer. The samples can then be analyzed in a laboratory to determine the average log reduction of the microbes in the test sites. To be classified as sanitized the results must indicate an average 5-log reduction in microbes. It will be appreciated, however, that for certain uses (for example, non-food contact surfaces) a 5-log reduction in microbes may not be necessary, and the system can be altered accordingly for example using lower power UV lamps, or irradiating the articles for a shorter period of time. For example, some articles may be required to be disinfected (classified as a 3-log reduction in bacteria), and the parameters of the system may be set accordingly. Alternatively, for certain uses a greater degree of sanitization may be required.

It should be understood that the examples and embodiments described herein are for illustrative purposes only and that various modifications or changes in light thereof will be suggested to persons skilled in the art and are to be included within the spirit and purview of this application. The invention can take other specific forms without departing from the spirit or essential attributes thereof.

What is claimed is:

1. An apparatus for sanitizing articles, comprising:
a conveyor for conveying articles through the apparatus;
an irradiation chamber surrounding the conveyor;
a plurality of UV lamps arranged in the irradiation chamber to irradiate with UV light articles conveyed through the irradiation chamber on the conveyor.

2. The apparatus according to claim 1, wherein the conveyor continuously conveys articles through the irradiation chamber.

3. The apparatus according to claim 1, wherein the UV lamps are arranged above and below the conveyor

4. The apparatus according to claim 1, wherein the conveyor has apertures to allow UV light to irradiate the bottom of articles placed thereon.

5. The apparatus according to claim 1, wherein the conveyor is selected from the group consisting of belt, mesh, hanging, plastic chain, and metal chain conveyors.

6. The apparatus according to claim 5, wherein the conveyor comprises a plurality of chain conveyors, each having chains located at different widths across the conveyor.

7. The apparatus according to claim 7, wherein each chain conveyor has at least two chains.

8. The apparatus according to claim 1, wherein the plurality of UV lamps are arranged in an ordered array.

9. The apparatus according to claim 8, wherein the array of UV lamps include lamps of different lengths.

10. The apparatus according to claim 8, wherein the array of UV lamps include lamps of different shapes.

11. The apparatus according to claim 1, wherein the UV lamps are elongated and define an axis, and at least one of the UV lamps is arranged with its axis perpendicular to the direction of the conveyor.

12. The apparatus according to claim 1, wherein the UV lamps are elongated and define an axis, and at least one of the UV lamps is arranged with its axis at an angle to the direction of the conveyor.

13. The apparatus according to claim 1, wherein at least one of the UV lamps includes a parabolic shaped reflector.

14. The apparatus according to claim 11, wherein the reflector is cooled, the cooling means selected from the group consisting of water-cooling, chillers, air conditioners, and high velocity air flow.

15. The apparatus according to claim 1, wherein the UV lamps are mercury vapor arc lamps.

16. The apparatus according to claim 1, wherein the articles are reusable articles.

17. The apparatus according to claim 16, wherein the articles are containers.

18. A method of sanitizing articles for re-use comprising the steps of:
checking articles for cleanliness;
washing only articles which are discovered to be below a predetermined standard of cleanliness; and
irradiating articles which are discovered to be above a predetermined standard of cleanliness with UV light in order to sanitize such articles without washing.

19. The method according to claim 18, further comprising removing debris from the articles.

20. The method according to claim 18 wherein the washed articles are irradiated with UV light after the washing step is completed.

21. The method according to claim 18 wherein the irradiation takes places in a sanitization chamber while the articles are continuously moved through the chamber by a conveyor.

22. The method according to claim 21, wherein the speed of the conveyor is variable to vary the UV dose given to the articles.

23. The method according to claim 21, wherein the intensity of the UV light is variable to vary the UV dose given to the articles.

24. The method according to claim 21, wherein the UV dose given to articles is measured by periodically passing a UV radiometer through the chamber.
